(19) United States
(12) Patent Application Publication
(10) Pub. No.: US 2006/0018847 A1
(43) Pub. Date: Jan. 26, 2006

(54) Active ingredient combinations of polyhexamethylenebiguanidine hydrochloride and distearyldimethylammonium chloride and preparations comprising said active ingredient combinations

(75) Inventors: Rainer Kroepke, Schenefeld (DE); Dagmar Zelle, Bliedersdorf (DE); Alexander Filbry, Hamburg (DE); Gunhild Hamer, Hamburg (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 11/090,693

(22) Filed: Mar. 25, 2005

Related U.S. Application Data
(63) Continuation of application No. PCT/EP02/10817, filed on Sep. 26, 2002.

(51) Int. Cl. A61K 7/42; A61K 31/155; A61K 31/14

(52) U.S. Cl. 424/59; 424/78.27; 514/635; 514/643

(57) ABSTRACT

The invention is an active ingredient combination comprising polyhexamethylenebiguanidine hydrochloride and distearyldimethylammonium chloride and a cosmetic or dermatological formulation comprising polyhexamethylenebiguanidine hydrochloride and distearyldimethylammonium chloride. The invention also includes methods of fighting or preventing dermatological harm comprising applying to the skin a cosmetic or dermatological formulation comprising polyhexamethylene biguanidine hydrochloride and distearyldimethylammonium chloride. The invention also includes methods of preventing decay of organic substances in a formulation, comprising adding to said formulation polyhexamethylene biguanidine hydrochloride and distearyidimethylammonium chloride.

### CROSS-REFERENCE TO RELATED APPLICATIONS

This is a continuation application of PCT/EP02/10817, filed Sep. 26, 2002, which claims the benefit of DE 101 47 186.6, filed Sep. 25, 2001.

### FIELD OF THE INVENTION

The present invention concerns active ingredient combinations comprising polyhexamethylene biguanidine hydrochloride and distearyidimethylammonium as well as the use of active ingredient combinations as agents against bacteria, mycota and viruses, in particular for preserving cosmetic and dermatological emulsions. In particular embodiments, the present invention concerns preparations, preferably cosmetic and dermatological preparations containing such substances.

### BACKGROUND OF THE INVENTION

Healthy warm-blooded organisms, in particular healthy human skin, are colonized by a wide variety of nonpathogenic microorganisms. These so-called microflora of the skin are not only harmless, but also represent an important form of protection as a defense against opportunistic or pathogenic microbes.

Bacteria belong to the prokaryotic single-celled organisms. They can be crudely distinguished according to their shape (ball, cylinder, bent cylinder) as well as the structure of their cell wall (gram-positive, gram-negative). The physiology of the organisms also allows for finer distinctions. There thus exist aerobic, anaerobic and facultatively anaerobic bacteria. Many individuals are of medical significance owing to their characteristic as pathogenic microbes, while others are completely harmless.

Substances effective against bacteria have been known for some time now. Nevertheless, many of these antibacterial agents are not suitable for all medical, let alone cosmetic, applications, as the metabolic functions of the warm-blooded organism, in other words the diseased patient, are also affected by the application in one way or another.

Gram-negative microbes are typically Escherichia coli, Pseudomonas species as well as enterobacteria such as Citrobacter freundii.

Even gram-positive microbes play a role in cosmetics and dermatology. Thus, for example, bacterial secondary infections are also of etiological significance besides other influences. One of the most important microorganisms associated with unclean skin is Staphylococcus aureus. This is responsible for the symptoms Impetigo follicularis, furunculosis, abscesses, sepsis, pemphigus neonatorum, toxic shock syndrome, food poisoning etc. In addition to this, examinations have revealed that a high percentage (over 90% of those affected) of neurodermatitis patients exhibited an, in part, extremely high content of Staphylococcus aureus on the skin.

In contrast to bacteria, fungi (also referred to mycota or mycobionts) belong to the eukaryotes. In contrast to the so-called prokaryotes (procytes), eukaryotes are living organisms whose cells (eucytes) have a cell nucleus separated from the remaining cytoplasm by a nuclear envelope and nuclear membrane. The cell nucleus contains the genetic information stored in chromosomes.

Typical representatives of the mycobionts include yeasts (Protoascomycetes), moulds (Plectomycetes), mildews (Pyrenomycetes), false mildews (Phycomycetes) and mushrooms or toadstools (Basidiomycetes).

Fungi do not belong to the Plant Kingdom, but like plants have a cell wall as well as vacuoles filled with cell sap and plasma streaming clearly visible microscopically. They do not contain any photosynthetic pigments and are C-heterotroph. They grow under aerobic conditions and obtain energy through oxidation of organic substances. Several representatives, for example yeasts, are facultative anaerobes and capable of obtaining energy through fermentation processes.

Dermatomycoses are diseases in which certain species of fungi, in particular dermatophytes, penetrate the skin and hair follicles. Typical symptoms of dermatomycoses are vesicles, exfoliation, fissures and erosion, usually accompanied by itching or allergic eczema.

Dermatomycoses can essentially be divided into four groups: Dermatophyes (e.g. epidermophytosis, favus, microsporosis, trichophytosis), yeast mycoses (e.g. pityriasis and other mycoses caused by pityrospores, Candida infections, blastomycosis, Busse-Buschke disease, torulosis, piedra alba, torulopsosis, trichosporosis), mould mycoses (e.g. aspergilosis, cephalosporiosis, phycomycosis and scopulariopsis) and system mycoses (e.g. chromomycosis, coccidiomycosis (Posada's disease) or histoplasmosis).

The pathogenic microbes typically include Candida species from the yeast group (e.g. Candida albicans) and those of the Pityrosporum family. Pityrosporum species, in particular Pityrosporum ovale, are responsible for skin diseases such as Pityriasis versicolor, Seborrhea in the forms Seborrhea oleosa and Seborrhea sicca which are primarily manifested as Seborrhea capitis (dandruff), seborrheic eczema and Pityrosporum folliculitis. An involvement of Pityrosporum ovale in the development of psoriasis is currently under discussion in the medical world.

All regions of the human skin can be affected by dermatomycoses. Dermatophytes almost exclusively attack the skin, hair and nails. Yeast mycoses regularly extend to the entire organ systems.

Those areas of the skin in which humidity and warmth can accumulate on account of clothing, jewelry or shoes are affected particularly frequently. Athlete's foot represents one of the most familiar and widespread of these. Fungal diseases in the nail areas of the finger and foot are also particularly unpleasant.

Moreover, super-infections of the skin by fungi and bacteria are not uncommon.

If a primary infection is extant, i.e. if a new infection occurs with a high number of microbes for one or several pathogens (for example staphylococci—often however nonpathogenic agents—for example Candida albicans), “super-infection” of the affected skin can occur if unfavorable influences coincide. The normal microflora of the skin (or another organ of the body) are overwhelmed by the secondary pathogens in this event.

Depending on the microbe concerned—such super-infections can be manifested in favorable cases as unpleasant skin appearances (itching, unattractive external appearance). In unfavorable cases, however, they can lead to large-scale destruction of the skin, in the worst situation even the death of the patient.

Such super-infections are observed for a wide variety of dermatological diseases, for example eczema, neurodermitis, acne, seborrheic dermatitis or psoriasis. Even many medical and therapeutic measures, e.g. radiography or chemotherapy for tumor diseases, medicinally-induced immunosuppression caused as a side-effect or systemic treatment with antibiotics, as well as external chemical or physical influences (e.g. environmental pollution, smog), promote the occurrence of super-infections of the external and internal organs, in particular the skin and the mucous membrane.

Although it is perfectly possible to fight super-infections with antibiotics, such substances usually have the disadvantage of unpleasant side effects. Patients are often allergic to penicillin for example, as result of which a corresponding treatment would be ruled out in such cases.

Moreover, topically administered antibiotics have the drawback of not only removing secondary pathogens from the skin, but also have an adverse effect on the purely physiological skin flora, with the result that the natural healing process is slowed down again.

The aim of the present invention was to remedy the disadvantage of the prior art and provide substances and preparations containing such substances through the use of which super-infections an be cured, whereby the physiological skin flora does not suffer any significant side effects.

In contrast to the prokaryotic and eukaryotic cellular organisms, viruses represent biological structures which require a host cell for biosynthesis. Extracellular viruses (also referred to a “virions”) consist of a single or double stranded nucleic acid sequence (DNS or RNS) and a protein mantle (called the capsid), sometimes surrounded by an additional envelope containing lipids. The entire structure comprising nucleic acid and capsid is referred to as the nucleocapsid. Viruses were originally classified according to traditional and clinical criteria, but nowadays they are generally classified according to their structure, morphology and, in particular, the nucleic acid sequence.

Medically important virus genera are typically influenza viruses (the Orthomyxoviridae family), Lyssaviruses (e.g. rabies, Rhabdovirus family), Enteroviruses (e.g. Hepatitis-A, Picornaviridae family), Hepadnaviruses (Hepatitis-B, Hepadnavirus family).

Virucides, i.e., substances capable of killing viruses do not actually exist in the strict sense of the term, as viruses do not have their own metabolism. Pharmacological interventions without damage to the cells that are not affected are, however, difficult. Possible mechanisms of action in the fight against viruses are primarily the destruction of their replication, e.g., by blocking the enzymes in the host cell important for their replication. Furthermore, the release of viral nucleic acids into the host cell can also be prevented. Within the scope of the disclosure presented here, the terms “antiviral” or “effective against viruses”, “virucide” or similar refer to the property of a substance enabling it to protect a single or multi-cellular organism against the harmful effects of a viral infection, be it prophylactic or therapeutic, regardless of what the actual mechanism of action of the substance in individual cases is.

Substances effective against viruses which do not damage the host organism, or do not do so to an unjustifiable degree, are absent in the prior art.

A further aim of the present invention was therefore to find substances which effectively protect a single-celled or multi-cellular organism against the detrimental effects of a viral infection, be it prophylactic or therapeutic.

Preservatives are antimicrobial substances which are added to a product (food and luxury items, pharmaceutical, cosmetic or also chemical-technical preparations) during the production process in small amounts (usually between approx. 0.0005% and 1% active content, depending on the product). Preservatives are intended to protect products from contamination by microorganisms, in particular from detrimental changes caused by microbes, during production, storage and consumption.

The following requirements are essentially demanded of a preservative: It must be sufficiently antimicrobial, as well as capable of being applied technically and safely in respect to health. The finished preparation—the commercial product—must also be harmless to health. Here it is necessary to consider that microorganisms, e.g., in cosmetic preparations, can be present primarily as a result of production or can enter the cosmetic preparation secondarily on account of the consumer.

It must therefore be ensured that the finished product is also safe throughout the entire period of consumption.

Preservatives authorized in food technology—such as those listed in the following table—can also be used effectively for the production of pharmaceutical or cosmetic products.

| | |
|---|---|
| E200 | Sorbic acid |
| E201 | Sodium sorbate |
| E202 | Potassium sorbate |
| E203 | Calcium sorbate |
| E210 | Benzoic acid |
| E211 | Sodium benzoate |
| E212 | Potassium benzoate |
| E213 | Calcium benzoate |
| E214 | Ethyl p-hydroxybenzoate |
| E215 | Sodium ethyl p-hydroxybenzoate |
| E216 | Propyl p-hydroxybenzoate |
| E217 | Sodium propyl p-hydroxybenzoate |
| E218 | Methyl p-hydroxybenzoate |
| E219 | Sodium methyl p-hydroxybenzoate |
| E220 | Sulfur dioxide |
| E221 | Sodium sulfite |
| E222 | Sodium hydrogen sulfite |
| E223 | Sodium metabisulfite |
| E224 | Potassium metabisulphite |
| E226 | Calcium sulfite |
| E227 | Calcium hydrogen sulfite |
| E228 | Potassium hydrogen sulfite |
| E230 | Biphynel (Diphenyl) |
| E231 | Orthophenyl phenol |
| E232 | Sodium orthophenyl phenyl |
| E233 | Thiabendazole |
| E235 | Natamycin |
| E236 | Formic acid |
| E237 | Sodium formate |
| E238 | Calcium formate |
| E239 | Hexamethylenetetramine |
| E249 | Potassium nitrite |
| E250 | Sodium nitrite |
| E251 | Sodium nitrate |
| E252 | Potassium nitrate |
| E280 | Propionic acid |
| E281 | Sodium propionate |
| E281 | Calcium propionate |
| E283 | Patassium propionate |
| E290 | Carbon dioxide |

The following also represent preservatives or preservative additives commonly used in the cosmetics industry: Dibromodicyanobutane (2-bromo-brommethylglutarodinitrile), 3-iodo-2-propynyl butylcarbamate, 2-bromo-2-nitro-propane-1,3-diol, imidazolidinyl urea, 5-chloro-2-methyl-4-isothiazolin-3-on, 2-chloracetamide, benzalkonium chloride, benzyl alcohol.

In addition to this, phenylhydroxyalkylethers, in particular the compound familiar under the designation phenoxyethanol, were used as preservatives on account of their bactericidal and fungicidal effects on a number of micro-organisms.

Polyhexamethylene biguanidine hydrochloride is also familiar in itself as a preservative for O/W and W/O emulsions. Polyhexamethylene biguanidine hydrochloride with the CTFA designation “Polyaminopropyl Biguanide” (CAS NO. 28757-47-3) is distinguished by the following formula: 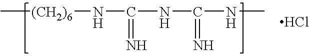
and is available from Zeneca Biocides under the trade name ARLAGARD® E.

ARLAGARD® E is a colorless to slightly yellowish, odorless, aqueous cationic solution with a wide antimicrobial spectrum (effective against gram-positive and gram-negative bacteria as well as yeasts and fungi).

Most preservatives proposed or envisaged for preservation have a bacteriostatic and fungistatic effect, occasionally also bactericidal and fungicidal. They should be odorless and tasteless and, if possible, soluble, non-toxic, skin-compatible and sufficiently effective in the dose applied.

In order to be effective, the preservatives must be dissolved in the raw material or auxiliary agent to be preserved. As most preservatives are more fat-soluble than water-soluble, it must be considered that, for example, in an emulsion whose aqueous phase is to be preserved, the preservative incorporated in the aqueous phase migrates to the fat phase during the course of storage, and hence calls into question the preservation of the aqueous phase. This poses a problem for the preservation of oil-in-water formulations in particular.

Although no sterility is generally required for a cosmetic preparation, it must be free of pathogenic microbes and protected against microbially induced changes.

It is important to consider that the various emulsion types, aqueous solutions, suspensions etc. require a different preservation, that the preservative effect of individual preservatives depends on the composition and the physical properties of the preparation to be preserved, that interactions between the preservative, the active agent and the auxiliary agents are to be expected, that various active or auxiliary agents can adsorb and hence potentially inactivate preservatives, that in particular hydrocolloids contained in the preparation can impede preservatives in their antimicrobial activity, depending on the concentration, and finally that—also depending on the concentration and type of preservative—the stratum corneum adsorbs the preservative, with the result that permeation and absorption of the preservative may then occur.

### SUMMARY OF THE INVENTION

It was surprisingly discovered, and herein lies the solution to all these objectives, that active agent combinations comprising polyhexamethylene biguanidine hydrochloride and distearyldimethylammonium chloride as well as the use of such active agent combinations as antibacterial, antimycotic or antiviral agents remedies the prior art.

The active agent combinations according to the invention astonishingly act in a synergistic manner, i.e., with an overadditive effect in relation to the individual components. This was not to be expected in particular on account of the fact that distearyldimethylammonium chloride itself exhibits no antimicrobial effect.

The active agent combinations according to the invention also make substances available which are effective against gram-positive and/or gram-negative bacteria without entailing an untenable effect on the health of the user.

Distearyidimethylammonium chloride (also: dimethyldioctadecylammonium chloride, quarternium-5) with the CTFA designation “Distearyidimonium Chloride” is a quaternary ammonium salt which is distinguished by the following structural formula:

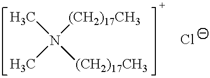

Especially advantageous is the use of the active agent combinations according to the invention as a preservation system for cosmetic or dermatological oil-in-water formulations, in particular for cosmetic or dermatological O/W emulsions.

It has surprisingly been revealed that the active agent combinations used according to the invention—in an overadditive manner to be precise—prevent the growth of gram-positive and gram-negative bacteria, mycobionts and viruses.

In particular, the active agent combinations used according to the invention are capable of preventing the growth of gram-positive bacteria, especially the staphylococcus species, namely Staphylococcus areus.

Moreover, it was astounding that the active agent combinations used according to the invention are particularly effective against neurodermatitis and that they prevent the development of typical recurrences of neurodermatitis. A preferred embodiment of the present invention is therefore represented by the formulations to be used against neurodermatitis.

Finally, it has been revealed that the active agent combinations used according to the invention can prevent the decay of organic substances, in particular cosmetic and dermatological preparations, as a result of attack by gram-positive and gram-negative bacteria, mycobionts and viruses, if they are added to these preparations.

Corresponding to the invention is a process for fighting mycobionts characterized by the fact that the active agent combinations used according to the invention, if possible in a suitable cosmetic or dermatological carrier, are brought into contact with the area contaminated with mycobionts; as well as a process for the protection of organic products against attack by mycobionts, characterized by the fact that the active agent combinations used according to the invention are added to these products in an effective concentration.

The prior art did not consequently provide any reference whatsoever concerning the use as an antimycotic mechanism in accordance with the invention.

### DETAILED DESCRIPTION OF THE PREFERRED EMBODIMENTS

Corresponding to the invention, the active agent combinations are preferably used in cosmetic or dermatological compositions with a content of 0.0005 to 50.0 weight %, in particular 0.01 to 20.0 weight % in relation to the total weight of the composition. Advantageously, the compositions contain 0.02 weight %, preferably 0.02 to 5.0 weight % of the active agent combinations used according to the invention, while especially preferred is 0.5 to 3.0 weight %, respectively in relation to the total weight of the composition.

The active agent combinations used according to the invention can be incorporated without any difficulty in current cosmetic or dermatological formulations, preferably pump sprays, aerosols sprays, cremes, ointments, tinctures, lotions, nail care products (e.g. nail varnish, nail varnish remover, and nail balsam) and similar.

Preparations extant as emulsions according to the present invention contain one or more O/W emulsifiers as well as, possibly, additional co-emulsifiers. Should the emulsions contain additional O/W emulsifiers which may be present in the preparation beside the distearyldimethylammonium chloride, these (one or more compounds) can, for example, be advantageously selected from the group of polyethoxylated and/or polypropoxylated emulsifiers.

The co-emulsifier or co-emulsifiers are preferably selected from the group of fatty alcohols with 14 to 22 carbon atoms, in particular from the group of fatty alcohols with 16 to 18 carbon atoms such as stearyl alcohol, cetyl alcohol, cetyl stearyl alcohol (cetearyl alcohol).

It is also possible, and possibly advantageous, to combine the active agent combinations used according to the invention with other active agents, for example with other substances with antimicrobial, antimycotic or antiviral effects.

If the active agent combinations corresponding to the invention are used as active agents for the preservation of organic materials, another or several other preservatives selected from the substances with the E-numbers E-200 to E-299 can additionally be used advantageously and/or another or several other preservatives selected from the substances dibromodicyanobutane (2-bromo-brommethylglutarodinitrile), phenoxyethanol, 3-iodo-2-propynyl butylcarbamate, 2-bromo-2-nitro-propane-1.3-diol, imidazolidinyl urea, 5-chloro-2-methyl-4-isothiazolin-3-on, 2-chloracetamide, benzalkonium chloride, benzyl alcohol.

It is possible, albeit not necessary, to buffer the compositions. A pH range of 4.5 to 7.5 is advantageous. It is especially preferably to select the pH value in a range of 5.0 to 6.5.

The cosmetic and/or dermatological formulations according to the invention can be composed as usual and serve the treatment of skin and/or hair within the scope of a dermatological treatment or a treatment within the scope of body care cosmetics. They can also, however, be used in makeup products in decorative cosmetics.

The cosmetic and/or dermatological formulations according to the invention are applied in the usual method for cosmetics and skin care products, i.e. to the skin and/or hair in a sufficient concentration.

Advantageous are those cosmetic and/or dermatological preparations which exist in the form of a sun protection agent. Preferably, these additionally contain at least one UVA filter and/or at least one UVB filter and/or at least one inorganic pigment.

Cosmetic preparations corresponding to the invention for protecting the skin against UV radiation can exist in various forms, as are normally used for these types of preparations. They can thus represent, for example, a solution, an emulsion of the type water-in-oil (W/O) or of the type oil-in-water (O/W), or multiple emulsions, for example of the type water-in-oil-in-water (WIOiw), a gel, a hydrodispersion, a solid pen or an aerosol.

The cosmetic preparations corresponding to the invention can contain cosmetic auxiliary agents, as are normally used in such preparations, for example preservatives, bactericides, antioxidants, perfumes, anti-foaming agents, colorings, pigments which have a coloring effect, thickening agents, surfactants, emulsifiers, plasticizing agents, moisturizing and/or moistening agents, fats, oils, waxes and other standard components of a cosmetic formulation such as alcohols, polyols, polymers, foam stabilizers, electrolytes, organic solvents or silicone derivatives.

If the cosmetic or dermatological preparation represents a solution or lotion, the following solvents can be used:
Water or aqueous solutions;
Oils, such as triglyceride of capric or caprylic acid, but preferably castor oil;
Fats, waxes and other natural and synthetic fat bodies, preferably ester of fatty acids with alcohols comprising a low C-number, e.g. with isopropanol, propylenglycol or glycerin, or ester of fatty alcohols with alkanoic acids comprising a low C-number or with fatty acids;
Alcohols, diols or polyols comprising a low C-number, as well as those of ether, preferably ethanol, isopropanol, propylenglycol, glycerin, ethylene glycol, ethylene glycol monoethyl ether, ethylene glycol monobutyl ether, propylene glycol monomethyl ether, propylene glycol monoethyl ether, propylene glycol monobutyl ether, diethylene glycol monomethyl ether or diethylene glycol monoethyl ether and analogous products.

In particular, mixtures of the above solvents are used. Water can be a further component for alcoholic solvents.

Particularly favorable preparations are also attained if antioxidants are used as additives or active agents. Corresponding to the invention, the preparations preferably contain one or more antioxidants. All antioxidants suitable or normal for cosmetic and/or dermatological applications can be used as favorable, albeit optional, antioxidants.

Particularly advantageous in the sense of the present invention is the use of water-soluble antioxidants, such as vitamins, e.g., ascorbic acid and its derivates such as D-biotin, natural and synthetic isoflavanoids, alpha-glycosyl rutin, Panthenol, Aloe vera.

The amount of antioxidants (one or several compounds) in the preparations is preferably 0.001 to 30 weight %, particularly preferred 0.05 to 20 weight %, especially 0.1 to 10 weight %, in relation to the total weight of the preparation.

It is particularly advantageous if the cosmetic preparations according to the present invention contain cosmetic or dermatological agents, whereby the preferred active agents are antioxidants which can protect the skin from oxidative stress.

Further advantageous active agents are natural agents and/or derivatives such as phytoene, carnitine, carnosine, creatine, taurine and/or β-alanine.

The cosmetic preparations according to the invention can contain cosmetic auxiliary agents, as are commonly used in such preparations, e.g. preservatives, bactericides, substances with a deodorant effect, antiperspirants, insect repellants, vitamins, anti-foaming agents, colorings, pigments with colorizing effect, thickening agents, plasticizing agents, moisturizing and/or moistening agents, fats, oils, waxes or other standard components of a cosmetic formulation such as alcohols, polyols, polymers, foam stabilizers, electrolytes, dihydroxyacetone, organic solvents or silicone derivatives.

Preparations corresponding to the invention can also contain substances which adsorb UV radiation in the UVB spectrum, whereby the total amount of the filter substances is e.g. 0.1 weight % to 30 weight %, preferably 0.5 to 10 weight %, especially 1.0 to 6.0 weight %, in relation to the total weight of the preparations, in order to provide cosmetic substances which protect the skin or hair against the entire spectrum of ultraviolet radiation. They can also serve as sun protection agents for the hair.

Correspondingly, the preparations as defined by the present invention preferably contain at least one further UVA, UVB and/or broad-spectrum filter substance. Although not necessary, the formulations can, if required, also contain one or more organic and/or inorganic pigments as UV filter substances which can occur in the water and/or oil phase.

Preferred inorganic pigments are metal oxides and/or other metal compounds that are of low solubility or insoluble in water. In particular oxides of titanium (TiO₂), zinc (Zn₂O), iron (e.g. FeO₃), zirconium (ZrO₂), silicon (SiO₂), manganese (e.g. MnO), aluminum (Al₂O₃), cerium (e.g. Ce₂O₃), mixed oxides of the corresponding metals as well as blends of such oxides and sulfate of barium (BaSO₄).

The pigments can also advantageously be applied according to the invention in the form of more commercially available oily or aqueous predispersions. Dispersing agents and/or solubilizing agents can also advantageously be added to these predispersions.

The pigments can favorably be surface treated (coated) in accordance with the invention, whereby for example a hydrophilic, amphiphilic, or hydrophobic character is to be formed or retained. This surface treatment can involve providing these pigments with a thin hydrophilic and/or hydrophobic inorganic layer by way of a familiar process. The various surface coatings can also contain water within the scope of the present invention.

Inorganic surface coatings within the scope of the present invention can consist of aluminum oxide (Al₂O₃), aluminum hydroxide Al(OH)₃ or aluminum oxide hydrate (also: Alumina, CAS No.: 1333-84-2), sodium hexametaphosphate (NaPO₃)₆, sodium metaphosphate (NaPO₃)ₙ, silicon oxide (SiO₂) (also: Silica, CAS No.: 7631-86-9), or iron oxide (Fe₂O₃). These inorganic surface coatings can occur alone or in combination and/or in combination with organic coating materials.

Organic surface coatings within the scope of the present invention can consist of vegetable or animal aluminum stearate, vegetable or animal stearic acid, lauric acid, dimethylpolysiloxane (also: Dimethicone), methylpolysiloxane (Methicone), Simethicone (a mixture of dimethylpolysiloxane with an average chain length of 200 to 350 dimethylsiloxane units and silica gel) or alginic acid. These organic surface coatings can occur alone, in combination and/or in combination with inorganic coating materials.

Zinc oxide particles and predispersions of zinc oxide particles suitable according to the invention are available under the following commercial names from the companies cited:

| Commercial name | Coating | Manufacturer |
|---|---|---|
| Z-Cote HP1 | 2% Dimethicone | BASF |
| Z-Cote | / | BASF |
| ZnO NDM | 5% Dimethicone | H&R |

Suitable titanium oxide particles and predispersions of titanium oxide particles are available under the following commercial names from the companies cited.

| Commercial name | Coating | Manufacturer |
|---|---|---|
| MT-100TV | Aluminum hydroxide/ | Tayca Corporation |
| | stearic acid | |
| MT-100Z | Aluminum hydroxide/ | Tayca Corporation |
| | stearic acid | |
| Eusolex T-2000 | Alumina/Simethicone | Merck KgaA |
| Titanium dioxide T805 | Octyltrimethylsilane | Degussa |
| (Uvinul TiO₂) | | |

An advantageous organic pigment within the scope of the present invention is the 2,2′-methylene-bis-(6-(2H-benzotrialzol-2-yl)-4-(1,3,3,3-tetramethyl-butyl)-phenol) [INCl:Biscotyltriazol], which is available from CIBA-Chemikalian GmbH under the commercial name TINOSORB®.

Advantageous UVA filter substances within the scope of the present invention are dibenzoylmethane derivates, in particular the 4-(tert-Butyl)-4′-methoxydibenzoylmethane (CAS No. 70356-09-1) which is sold by Givaudan under the brand name PARSOL® 1789 and by Merck under the commercial name EUSOLEX® 9020.

Advantageous further UV filter substances within the scope of the following invention are sulfonated, water-soluble UV filters, for example:
Phenylene-1,4-bis-(2-benzimidazyl)-3,3′-5,5′-tetrasulfonic acid and its salts, In particular the corresponding sodium, potassium or triethanol ammonium salts, in particular the phenylene-1,4-bis-(2-benzimidazyl)-3,3′-5,5′-tetrasulfonic acid bis sodium salt with the INCI designation Bisimidazylate (CAS No.: 180898-37-7) which is available for example from Haarmann & Reimer under the commercial name Neo Heliopan;
Salts of the 2-Phenylbenzimidazole-5-sulfonic acid, such as its sodium, potassium or its triethanol ammonium salts as well as the sulfonic acid itself with the INCI designation Phenylbenzimidazole sulfonic acid (CAS No.: 27503-81-7), which is available for example from Merck under the commercial bame Eusolex 232 or from Haarmann & Reimer under the commercial name Neo Heliopan;
1,4-di(2-oxo-10-sulfo-3-bornylidenmethyl)-benzol (also: 3.3′-(1,4-phenylene-dimethylene)-bis-(7,7-dimethyl-2-oxo-bicyclo-[2-2-1]hept-1-ylmethane sulfonic acid) and its salts (in particular the corresponding 10-sulfato compounds, in particular the corresponding sodium, potassium or triethanol ammonium salts), which is also designed as Benzol-1.4-di(2-oxo-3-bornylidenmethyl-10-sulfonic acid). Benzol-1.4-di(2-oxo-3-bornylidenmethyl-10-sulfonic acid has the INCI designation Terephthalidene Dicamphor Sulfonic Acid (CAS No.: 90457-82-2) and is available for example from the company Chimex under the commercial name Mexoryl SX;
Sulfonic acid derivatives of 3-benzylidene camphor, such as 4-(2-oxo-3-bornylidenmethyl)benzol sulfonic acid, 2-methyl-5-(2-oxo-3-bornylidenmethyl) sulfonic acid and their salts.

Advantageous UV filters within the scope of the present invention are also the so-called broad-spectrum filters, i.e. filter substances which absorb both UVA and UVB radiation.

Advantageous broad-spectrum filters are typically triazine derivatives, such as:
2,4-bis-{[2-ethyl-hexyloxy)-2-hydroxyl]-phenyl}-6-(4-methoxyohenyl)-1,3,5-triazine (INCI: Aniso Triazine), which is available from CIBA Chemikalien GmbH under the commercial name TINOSORB®;
Dioctylbutzlamidotriazone (INCI: Dioctylbutzlamidotriazone), which is available from Sigma 3V under the commercial name UVASORB HEB;
4,4′.4″-(1.3.5-triazine-2.4.6-triyltriimino)-tris-benzoic acid tris(2-ethylhexylester), synonym: 2,4,6-tris-[anilino-(p-carbo-2′-ethyl-1′-hexyloxy)]-1,3,5-triazine (INCI: Octyl Triazone), which is available from BASF under the product designation UVI-NUL® T 150.

An advantageous broad-spectrum filter within the scope of the present invention is 2,2′-methylene-bis(6-(2H-benzotriazol-2-yl)-4-(1,1,3,3-tetramethyl butyl)-phenol, which is available from CIBA Chemikalien under the commercial name TINSORB® M.

An advantageous broad-spectrum filter within the scope of the present invention is also 2-(2H-benzotriazol-2-yl)-4-methyl-6-[2-methyl-3-[1,3,3,3-tetramethyl-1-[(trimethylsilyl)oxy]disiloxanyl]propyl]-phenol (CAS No.: 155633-54-8) with the INCI designation Drometrizole Trisiloxane.

The UV filters can be oil soluble or water soluble. Advantageous oil-soluble filter substances are for example:
3-benzylidene camphor derivatives, preferably 2-(4-methylbenzylidene) camphor, 3-benzylidene camphor;
4-aminobenzoic acid derivatives, preferably 4-(dimethylamino)-benzoic acid (2- ethylhexyl)ester, 4-(dimethylamino)-benzoic acid amylester;
2,4,6-trianilino-(p-carbo-2′-ethyl-1′-hexyloxy)-1,3,5-triazine;
Ester of benzylmalonic acid, preferably 4-methoxybenzylmalonic acid di(2-ethylhexyl)ester;
Ester of cinnamic acid, preferably 4-methoxy cinnamic acid (2-ethzlhexyl)ester, 4-methoxy cinnamic acid isopentylester;
Derivatives of benzophenones, preferably 2-hydroxyl-4-methoxybenzophenone, 2-hydroxy-4-methoxy-4′-methylbenzophenonme, 2,2′-dihydroxy-4-methoxybenzophenone as well as
UV filters bonded to polymers.

Advantageous water-soluble filter substances are for example: Sulfonic acid derivatives of the 3-benzylidene camphor, such as 4-(2-oxo-3-bornylidene methyl) benzene sulfonic acid, 2-methyl-5-(2-oxo-bornylidene methyl) sulfonic acid and their salts.

A further sun protection substance which can favorably be used according to the invention is ethylhexyl-2-cyano-3,3-diphenylacrylate (Octocrylene), which is available from BASF under the name UVINUL® N 539.

The list of UV filters cited which can be used advantageously within the scope of the present invention should, of course, not be restrictive.

Preparations according to the invention can also contain nonionic and/or amphoteric surfactants, especially if crystalline or microcrystalline solids such as inorganic pigments are to be incorporated in the preparations according to the invention.

Surfactants are generally classified according to the type and charge of the hydrophilic part of the molecule. Four groups can be distinguished hereby:
Anionic surfactants,
Cationic surfactants,
Amphoteric surfactants and
Nonionic surfactants.

Anionic surfactants generally exhibit carboxylate, sulfate or sulfonated groups as functional groups. In an aqueous solution they form negatively charged organic ions in an acidic or neutral environment. Cationic surfactants are almost exclusively characterized by the presence of a quaternary ammonium group. In aqueous solution they form positively charged organic ions in an acidic or neutral environment. Amphoteric surfactants contain both anionic and cationic groups and therefore behave as anionic or cationic surfactants in an aqueous solution, depending on the pH value. In highly acidic environments they possess a positive charge and in alkaline environments a negative charge. In the neutral pH range, on the other hand, they are zwitterionic, as the following example should make clear:
RNH₂⁺CH₂CH₂COOH X⁻(at pH=2) X⁻=any anion, e.g. Cl⁻
RNH₂⁺CH₂CH₂COO⁻(at pH=7)
RNHCH₂CH₂COO⁻B⁺(at pH=12) B⁺=any cation, e.g. Na⁺

Typical for nonionic surfactants are polyether chains. Nonionic surfactants do not form ions in an aqueous medium.

A. Anionic Surfactants

It is particularly advantageous within the scope of the present invention for preparations containing the active agent combinations according to the invention not to contain any anionic surfactants in order to prevent a spontaneous salification and hence an inactivation of the active agents.

B Cationic surfactants

Cationic surfactants the use of which is advantageous are:
1. Alkylamines,
2. Alkyl imidazoles,
3. Ethyloxylated amines and
4. Quaternary surfactants.
5. Esterquats

Quaternary surfactants contain at least one N-atom which is covalently bonded with 4 alkyl or acryl groups. This leads to a positive charge, independent of the pH value. Alkyl betaine, alkyl amidopropyl betaine and alkyl amidopropyl hydroxysulfain are advantageous. The cationic surfactants used according to the invention can also preferably be selected form the group of quaternary ammonium compounds, in particular benzyltrialkyl ammonium chlorides or bromides, for example benzyldimethylstearyl ammonium chloride, as well as alkyltrialkyl ammonium salts, for example Cetyltrimethylstearyl ammonium chloride or bromide, alkyldimethylhydroxyethylehtyl ammonium chlorides or bromides, dialkyldimethyl ammonium chlorides or bromides, alkylamidethyltrimethyl ammonium ether sulfates, alkylpyridinium salts, for example Lauryl- or Cetylpyrimidinium chloride, imidazoline derivatives and compounds with cationic character such as amine oxides, for example alkyldimethylamine oxides or alkylaminoethyldimethylamine oxides. Particularly advantageous is the use of Cetyltrimethyl ammonium salts.

C Amphoteric Surfactants

Amphoteric surfactants the use of which is advantageous are:
1. Acyl-/dialkyl ethylene diamine, for example sodium acylamphoacetate, disodium acylamphoacetate, sodium acylamphohydroxypropyl sulfonate, disodium acylamphodiacetate and sodium acylamphopropionate;
2. N-alkylamine acids, for example aminopropylalkylglutamide, alkylaminopropion acid, sodium alkylimidodipronionat and Lauroamphocarboxyglycinate.

D Nonionic Surfactants

Nonionic surfactants the use of which is advantageous are:
1. Alcohols;
2. Alkanomides, such as Cocamides MEA/DEA/MIPA;
3. Amine oxides, such as Cocamidipropylamine oxide;
4. Esters which result from the esterification of carboxylic acids with ethylene oxide, glycerin, sorbitan or other alcohols;
5. Ethers, for example ethoxylated/propoxylated alcohols, ethoxylated/propoxylated esters, ethoxylated/propoxylated glycerin esters, ethoxylated/propoxylated cholesterols, ethoxylated/propoxylated triglyceride esters, ethoxylated/propoxylated lanolin, ethoxylated/propoxylated polysiloxanes, propoxylated POE-ether and alkylpolyglycosides such as Laurylglucoside, Decylglycoside and Cocoglycosid;
6. Sucrose esters, sucrose ethers;
7. Polyglycerine esters, diglycerine esters, monoglycerine esters;
8. Methylglucose esters, esters of hydroxy acids

The use of a combination of cationic and/or amphoteric surfactants with one or more nonionic surfactants is also advantageous.

The surface-active substance can be present in the preparations according to the invention at a concentration between 1 and 95 weight %, in relation to the total weight of the preparations.

The lipid phase of the cosmetic or dermatological emulsions according to the invention can favorably be selected from the following substance group:
Mineral oils, mineral waxes;
Oils, such as triglyceride of capric or caprylic acid, also natural oils such as castor oil;
Fats, waxes and other natural and synthetic fat bodies, preferably ester of fatty acids with alcohols comprising a low C-number, e.g. with isopropanol, propylenglycol or glycerin, or ester of fatty alcohols with alkanoic acids comprising a low C-number or with fatty acids;
Alkyl benzoates;
Silicone oils such asdimethylpolysiloxanes, diethylpolysiloxanes, diphenylpolysiloxanes as well as mixed forms thereof.

The oil phase for the emulsions of the present invention is advantageously selected from the groups of esters comprising saturated and/or unsaturated, branched or unbranched alkane carboxylic acids with a chain length of 3 to 30 C-atoms and saturated and/or unsaturated, branched and/or unbranched alcohols with a chain length of 3 to 30 C-atoms, from the group of esters comprising aromatic carboxylic acids and saturated and/or unsaturated, branched and/or unbranched alcohols with a chain length of 3 to 30 C-atoms. Such ester oils can then advantageously be selected from the group isopropyl myristat, isopropyl palmitate, isopropyl stearate, isopropyl oleate, n-butyl stearate, n-hexyllaurate, n-decyl oleate, iso-octyl stearate, isononyl stearate, isononyl isononanoate, 2-ethylhexyl palmitate, 2-hexyldecyl stearate, 2-octyldodecyl palmitate, oleyl oleate, oleyl erucate, erucyl oleate, erucyl erucate as well as synthetic, semi-synthetic and natural mixtures of such esters, e.g. jojoba oil.

Moreover, the oil phase can also advantageously be selected from the group of branched and unbranched hydrocarbons and waxes, the silicone oils, the dialkyl ethers, the group comprising the saturated or unsaturated, branched or unbranched alcohols, as well as the fatty acid glycerides, namely the triglycerin esters of unsaturated and/or unsaturated, branched and/or unbranched alkane carboxylic acids with a chain length of 8 to 24, in particular 12 and 18 C-atoms. The fatty acid triglycerides can be advantageously selected from the group of synthetic, semi-synthetic and natural oils, for example olive oil, sunflower oil, Soya oil, peanut oil, rape oil, almond oil, palm oil, coconut oil, palm kernel oil and similar.

In addition to this, any blends of such oil and water components can also be used advantageously within the scope of the present invention. It may also be favorable to use waxes, for example cetyl palmitate, as sole lipid components of the oil phase.

The oil phase is advantageously selected from the group 2-ethylhexyl isostearate, octyldodecanol, isotridecyl isononanoate, isoeicosane, 2-ethylhexyl cocoate, C₁₂₋₁₅-alkylbenzoate, caprylic/capric acid triglyceride, dicaprylyl ether.

Particularly advantageous are mixtures of C₁₂₋₁₅-alkylbenzoate and 2-ethylhexyl isostearate, mixtures of C₁₂₋₁₅-alkylbenzoate and isotridecyl isononanoate as well as mixtures of C₁₂₋₁₅-alkylbenzoate, 2-ethylhexyl isostearate and isotridecyl isononanoate.

Of the carbohydrates, paraffin oils, squalane and squalene are advantageous within the scope of the present invention.

Advantageously, the oil phase can also exhibit a content of cyclic or linear silicone oils or can consist entirely of such oils, whereby however an additional content of other oil phase components is preferably used instead of the silicone oil or the silicone oils.

Corresponding to the invention, the use of cyclomethicon (e.g. decamethylcyclopentasiloxane) is to be considered advantageous. However, other silicone oils are also favorable within the scope of the present invention, for example, undecamethylcyclotrisiloxane, polydimethylsiloxane and poly(methylphenylsiloxane).

Particularly advantageous are also mixtures comprising cyclomethicon and isotridecyl isononanoate, as well as from cyclomethicon and 2-ethylhexyl isostearate.

The aqueous phase of the preparation according to the invention may favorably contain one or more thickening agents which can be advantageously selected from the group silicon dioxide, aluminum silicates, polysaccharides or their derivatives, e.g. hyaluronic acid, xanthan rubber, hydroxypropyl methyl cellulose.

The following examples should explain but not restrict the invention. The numerals refer to the weight % insofar as not otherwise indicated.

### EXAMPLES

### O/W Emulsions

| | Example number | | | | |
|---|---|---|---|---|---|
| | 1 | 2 | 3 | 4 | 5 |
| Distearyldimmonium | 1 | 2 | 3.5 | 4 | 5 |
| chloride | | | | | |
| Cetyl stearyl alcohol | 1 | | | 0.5 | |
| Cetyl alcohol | 1 | 2.5 | 2 | | 0.5 |
| Dimethylpolysiloxane | 1 | 1 | 1 | 3 | 5 |
| Night candle oil | 2 | | | 7.5 | |
| Mineral oil | 3 | 5 | 6 | | |
| Vaseline | | 4.5 | 4 | | |
| Iodopropynyl | | 0.2 | 0.2 | | |
| butylcarbamate | | | | | |
| Polyhexamethylene | 0.5 | 1 | 1 | 2 | 3 |
| biguanidine | | | | | |
| hydrochloride | | | | | |
| Glycerin | 10 | 15 | 12 | 25 | 8 |
| Behenyl alcohol | 1 | | | | 1 |
| Stearyl alcohol | 1 | 1 | | | 1 |
| Methylparaben | 0.1 | | | 0.1 | 0.4 |
| Benzyl alcohol | | 1 | | 0.5 | |
| Hydrated coconut | 2 | | | 1 | |
| fat glycerides | | | | | |
| Shea butter | | 2 | | 1 | |
| Butylene glycol | 1 | | | | 5 |
| dicaprylate/dicaprate | | | | | |
| Caprylic/Capric | | 4 | 3 | | 1 |
| triglyceride | | | | | |
| Ethylhexyl coconut | 3 | | | 5 | |
| fatty acid ester | | | | | |
| Octyldodecanol | | | 3 | | |
| Octamethyltetrasiloxane | | 1 | | 1 | 3 |
| Yeast flour | | 1.5 | 1 | 1 | |
| EDTA | 0.5 | | | 1 | |
| BHT | 0.03 | | | 0.05 | |
| Dicaprylyl carbonate | | 5 | | | 2 |
| Perfume | q.s. | q.s. | q.s. | q.s. | q.s. |
| Water | Ad 100 | Ad 100 | Ad 100 | Ad 100 | Ad 100 |
| | Example number | | | | |
| | 6 | 7 | 8 | 9 | 10 |
| Distearyldimmonium | 1 | 2 | 3.5 | 3.5 | 5 |
| chloride | | | | | |
| Cetyl stearyl alcohol | 2.5 | | 2.5 | 1.5 | 0.5 |
| Cetyl alcohol | 2.5 | 2 | | 1.5 | 0.5 |
| Dimethylpolysiloxane | 3 | | 1 | | |
| Night candle oil | | 15 | 12.5 | | 7.5 |
| Mineral oil | 10 | | | | 7.5 |
| Vaseline | 2 | 5 | 4 | | 1 |
| Iodopropynyl | 0.1 | 0.18 | 0.2 | | |
| butylcarbamate | | | | | |
| Polyhexamethylene | 2 | 1 | 1 | 2.5 | 5 |
| biguanidine | | | | | |
| hydrochloride | | | | | |
| Glycerin | 25 | 14 | 12 | 3 | 5 |
| Behenyl alcohol | 0.75 | | | | 0.5 |
| Stearyl alcohol | 3 | | | | 0.5 |
| Methylparaben | | | | 0.1 | 0.25 |
| Benzyl alcohol | 0.5 | | | 1.5 | |
| Hydrated coconut | 2 | | | 1 | 5 |
| fat glycerides | | | | | |
| Shea butter | 0.5 | | | 1 | |
| Butylene glycol | 2 | | | 2 | |
| dicaprylate/dicaprate | | | | | |
| Caprylic/Capric | | | | | 1 |
| triglyceride | | | | | |
| Ethylhexyl coconut | | | | | |
| fatty acid ester | | | | | |
| Octyldodecanol | | | | 3 | |
| Octamethyltetrasiloxane | 2 | | | 1 | 3 |
| Yeast flour | | 2 | | 2 | 5 |
| EDTA | | 0.5 | 0.2 | | 0.3 |
| BHT | | 0.03 | 0.05 | 0.04 | |
| Dicaprylyl carbonate | 5 | | | | |
| Perfume | q.s. | q.s. | q.s. | q.s. | q.s. |
| Water | Ad 100 | Ad 100 | Ad 100 | Ad 100 | Ad 100 |

That which is claimed:

1. An active agent combination, comprising polyhexamethylene biguanidine hydrochloride and distearyldimethylammonium chloride.

2. A cosmetic or dermatological formulation, comprising polyhexamethylene biguanidine hydrochloride and distearyldimethylammonium chloride.

3. The cosmetic or dermatological formulation as claimed in claim 2, further comprising at least one antioxidant.

4. The cosmetic or dermatological formulation as claimed in claim 2, further comprising at least one UV filter substance.

5. The cosmetic or dermatological formulation as claimed in claim 2, wherein said formulation is in the form of an emulsion.

6. The cosmetic or dermatological formulation as claimed in claim 2, wherein said formulation is in the form of an oil-in-water emulsion.

7. The cosmetic or dermatological formulation as claimed in claim 2, wherein said formulation is in the form of a water-in-oil emulsion.

8. The cosmetic or dermatological formulation as claimed in claim 2, comprising from 0.01 to 20% by weight in total of the combination of polyhexamethylene biguanidine hydrochloride and distearyldimethylammonium chloride, based on the total weight of the formulation.

9. The cosmetic or dermatological formulation as claimed in claim 8, comprising from 0.02 to 5% by weight in total of the combination of polyhexamethylene biguanidine hydrochloride and distearyidimethylammonium chloride, based on the total weight of the formulation.

10. The cosmetic or dermatological formulation as claimed in claim 9, comprising from 0.05 to 3% by weight in total of the combination of polyhexamethylene biguanidine hydrochloride and distearyidimethylammonium chloride, based on the total weight of the formulation.

11. A method of preventing the growth of bacteria, mycobionts, and viruses, comprising applying an active agent combination comprising polyhexamethylene biguanidine hydrochloride and distearyldimethylammonium chloride.

12. A method of fighting or preventing dermatological harm, comprising applying to the skin a cosmetic or dermatological formulation comprising polyhexamethylene biguanidine hydrochloride and distearyldimethylammonium chloride.

13. The method as claimed in claim 12, wherein said formulation comprises from 0.01 to 20% by weight in total of the combination of polyhexamethylene biguanidine hydrochloride and distearyldimethylammonium chloride, based on the total weight of the formulation.

14. The method as claimed in claim 12, wherein said formulation comprises from 0.02 to 5% by weight in total of the combination of polyhexamethylene biguanidine hydrochloride and distearyldimethylammonium chloride, based on the total weight of the formulation.

15. The method as claimed in claim 12, wherein said formulation comprises from 0.05 to 3% by weight in total of the combination of polyhexamethylene biguanidine hydrochloride and distearyldimethylammonium chloride, based on the total weight of the formulation.

16. The method as claimed in claim 12, for preventing or improving neurodermatitis.

17. The method as claimed in claim 12, for preventing the growth of or killing Staphylococcus aureus.

18. A method of preventing decay of organic substances in a formulation, comprising adding to said formulation polyhexamethylene biguanidine hydrochloride and distearyldimethylammonium chloride.

19. The method as claimed in claim 18, wherein said formulation comprises from 0.01 to 20% by weight in total of the combination of polyhexamethylene biguanidine hydrochloride and distearyldimethylammonium chloride, based on the total weight of the formulation.

20. The method as claimed in claim 18, wherein said formulation comprises from 0.02 to 5% by weight in total of the combination of polyhexamethylene biguanidine hydrochloride and distearyldimethylammonium chloride, based on the total weight of the formulation.

21. The method as claimed in claim 18, wherein said formulation comprises from 0.05 to 3% by weight in total of the combination of polyhexamethylene biguanidine hydrochloride and distearyldimethylammonium chloride, based on the total weight of the formulation.
